(19) United States
(12) Patent Application Publication
(10) Pub. No.: US 2006/0100563 A1
(43) Pub. Date: May 11, 2006

(54) Body joint liner

(76) Inventor: Robert Farrer Gilmour, Auckland (NZ)

Correspondence Address: YOUNG & THOMPSON, 745 SOUTH 23RD STREET, 2ND FLOOR, ARLINGTON, VA, 22202, US

(21) Appl. No.: 10/516,822

(22) PCT Filed: Jun. 17, 2003
(86) PCT No.: PCT/NZ03/00121

§ 371 Date: Oct. 5, 2005

(30) Foreign Application Priority Data
Jun. 18, 2002 (NZ) 519623

(51) Int. Cl. A61F 13/00; A61F 13/06

(52) U.S. Cl. 602/60; 602/65

(57) ABSTRACT

A body joint liner (1) comprises an inner face (3) and an outer face, the inner face has a channel (2) therein open (6) at one end and closed (5) at the other end, being of substantially constant width and depth. The inner face is shaped to the contours of the body joint. The liner might be attached to a hard plastic cover (10) via glue, tape or the like.

### TECHNICAL FIELD OF THE INVENTION

This invention relates to a liner particularly a body joint liner such as an ankle brace for human use.

### BACKGROUND OF THE INVENTION

There are a variety of known ankle brace designs of a type frequently known as clam shell ankle braces. These braces consist of a pair of shells with body joint liner inserts so that the construction can be attached to either side of for example the ankle joint. They are frequently fitted following common soft tissue ankle injuries, such as those known as inversion industry or odema (swollen tissue). Following the injury regaining ones health and fitness as quickly as possible is important. A contributing factor which influences the healing process is the ease with which tissue fluid drains from an injury site. Also muscle surrounding the body joint may be damaged and this can affect a person's balance. Existing braces provide a moulded foam liner with a straight channel running up the middle of the liner. This channel provides an area of lesser pressure and so encourages drainage of tissue fluid following the common inversion injury.

Proprioception refers to a sense of position for a body. In the soft tissues around a joint there are “sense of position receptors” which provide information to the brain which then in a reflex fashion instructs muscle to move to control positional movement. Although ligaments stabilise joints in a static sense, it is the dynamic stabilisation of joints achieved by the muscles that cross them, that is primarily in control. After an injury there is propriosceptive lag which makes the ankle more susceptible to a lack of stabilisation because the muscle reflex is less responsive.

It has been found that many of the known body joint liners fail to achieve satisfactory results in that the healing process which involves the movement of tissue fluids can be too slow and the liner can be too uncomfortable to wear due to high skin pressures.

### OBJECT OF THE INVENTION

It is an object of the present invention to provide a liner which will reduce the foregoing disadvantages in a simple yet effective manner or which will at least provide the public with a useful choice.

### STATEMENT OF THE INVENTION

Accordingly in one preferred form of the invention a liner is provided which has an inner face and an outer face, the inner face having therein a channel open at one end and closed at the other end, the channel being substantially of constant width and depth over at least a substantial portion of the length of the channel, and at least part of the inner face being shaped to substantially approximate the contours of a selected joint of the human body.

Preferably the liner is elongated, the channel running substantially axially along the liner.

Preferably the liner has a concavity therein, the closed end of the channel being at or about the concavity.

Preferably a substantially rigid cover is provided shaped to receive the outer face of the liner.

Preferably the cover and liner are adhered one to the other.

To those skilled in the art to which the invention relates, many changes in construction and widely differing embodiments and applications of the invention will suggest themselves without departing from the scope of the invention as defined in the appended claims. The disclosures and the description herein are purely illustrative and are not intended to be in any sense limiting.

### BRIEF DESCRIPTION OF THE DRAWINGS

The invention consists of the foregoing and also envisages constructions of which the following gives examples.

One preferred form of the present invention will now be described with reference to the accompanying drawings in which,

FIG. 1 is an inside perspective view of a liner according to one preferred form of the invention,

FIG. 2 is a perspective view of the outer face of the liner of FIG. 1,

FIG. 3 is a longitudinal cross section through the liners of FIGS. 1 and 2,

FIG. 4 is an internal perspective view of a cover intended for use with the liner of FIG. 1,

FIG. 5 is an external view of the cover of FIG. 4, and

FIG. 6 is a longitudinal cross section view of the cover of FIGS. 4 and 5.

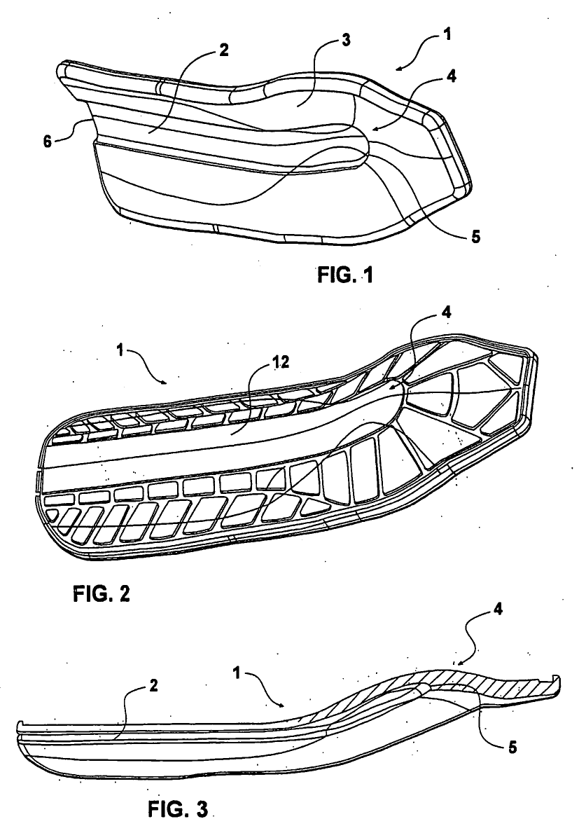

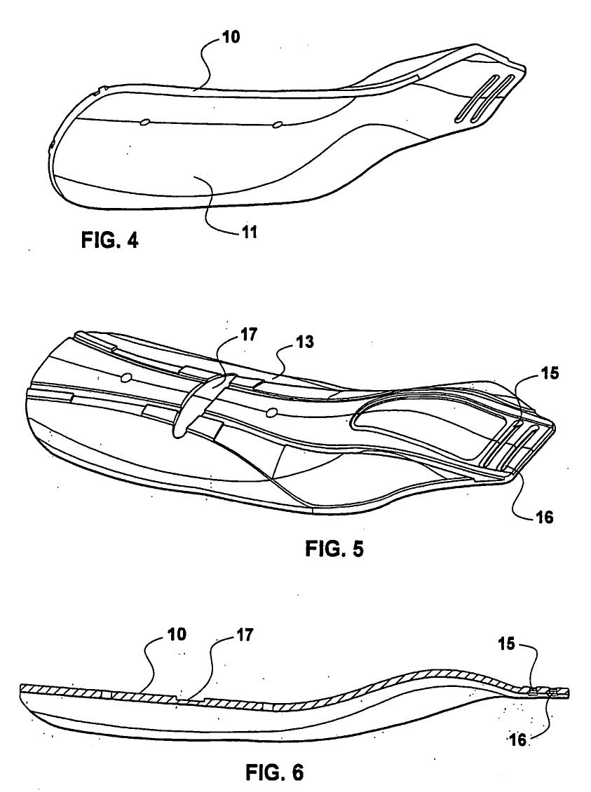

### DETAILED DESCRIPTION OF THE DRAWINGS

Referring to the drawings, a liner 1 is provided which may be formed of a foamed plastics material so as to provide the necessary “softness” and not to provide substantial hardness or hard edges against the body of a user.

A channel 2 is provided on the inner face 3 of the liner. The inner face 3 is shaped over at least part thereof to the contours of a selected joint of a human, in particular, the ankle. The channel 2 is desirably of substantially constant width and may be of substantially constant depth along substantially all its length, however there may be some taper towards the upper end 6 or extra depth at a concavity 4. The concavity 4 is provided in the liner 1 shaped so as to be a fit over the inner or outer ankle parts for example of a user. The channel 3 is closed at end 5 and end 5 is positioned substantially at the centre point of the concavity at 4. The channel is open at end 6 which is the upper end in use.

In transverse cross section the liner 1 is also desirably formed to a curve that will generally fit against the leg of a user above the ankle joint. The channel 3 is in addition to the general curve to fit the leg of the user. The dimensions of the channel do not have to be precise but by way of example the channel could be about 25-30 mm wide in a total line width of the liner of about 85-90 mm. A suitable depth could be about 4 mm deep at its deepest point in the concavity and about 1 to 2 mm adjacent the open channel end and along most of the length of the channel 3.

The liner 1 desirably is engaged by a cover 10 which may be formed of a plastics material desirably a hard plastics material. The cover 1 is desirably shaped so that the inner surface 11 thereof will substantially fit the outer surface 12 of the liner 1. Thus the liner 1 may be placed inside the cover and is desirably engaged therewith for example by an adhesive such as glue, double sided tape or the like.

The outer face 13 of the cover 10 may be formed to any desirable shape and may for example include slots 15 and 16 able to receive fixing devices and other constructions such as a depression 17 to allow identification material to be placed therein.

In use the liner 1 is engaged with a cover 10 and a pair of such constructions may be provided about a user's ankle. The construction may be positioned for example by straps or a stocking or in other ways so that the channel 2 has its end 5 adjacent an ankle. The channel 2 provides a channel by which the fluids caused by injury for example may drain away from the injury site adjacent the ankle. As the construction is relatively soft there is little or no effect on the sense of position receptors in the vicinity of and about the ankle, which is desirable.

What we claim is:

1. A liner having an inner face and an outer face, the inner face having therein a channel open at one end and closed at the other end, the channel being substantially of constant width and depth over at least a substantial portion of the length of the channel, and at least part of the inner face being shaped to substantially approximate the contours of a selected joint of the human body.

2. A liner as claimed in claim 1 wherein the liner is elongated, and the channel runs substantially axially along the liner.

3. A liner as claimed in claim 1, wherein the liner has a concavity therein, the closed end of the channel being at or about the concavity.

4. A liner as claimed in claim 1 further including a substantially rigid cover and shaped to receive the outer face of the liner.

5. A liner and cover as claimed in claim 4 wherein the cover and liner are adhered one to the other.
